(19) United States
(12) Patent Application Publication
(10) Pub. No.: US 2026/0085026 A1
(43) Pub. Date: Mar. 26, 2026

(54) CARBON EMISSION REDUCTION IN PROCESS UNIT BY INCREASED HYDROCARBON RECOVERY FROM NET GAS STREAM

(71) Applicant: UOP LLC, Des Plaines, IL (US)

(72) Inventors: Srinivasan Ramanujam, Haryana (IN); Harsh Satishbhai Patel, Gujarat (IN); Avijit Basu, New Delhi (IN)

(21) Appl. No.: 19/241,562

(22) Filed: Jun. 18, 2025

(30) Foreign Application Priority Data
Sep. 23, 2024 (IN) 202411071815

(51) Int. Cl. C07C 7/11; B01D 53/14; B01D 53/22; C01B 3/38; C01B 3/50

(52) U.S. Cl. CPC C07C 7/11; B01D 53/1443; B01D 53/229; C01B 3/38; C01B 3/501; B01D 2256/24; B01D 2257/108; C01B 2203/0227; C01B 2203/0405; C01B 2203/048; C01B 2203/062; C01B 2203/1235

(57) ABSTRACT

Processes for recovering hydrogen and hydrocarbons from a process stream are described. The processes incorporate a membrane separation unit to reject hydrogen preferentially from the net gas and recover the C₃₊ hydrocarbons from the hydrogen depleted retentate stream from the membrane unit using a retentate absorber. The invention eliminates the need for a large tail gas compressor package, while still achieving the target C₃₊ recovery of 85-90% or more with a smaller refrigeration package in the net gas section and membrane and retentate absorber equipment. It does not require any additional compressors, resulting in a significant reduction in plot space and power consumption.

### RELATED APPLICATIONS

This application claims priority to Indian Provisional Patent Application Ser. No. 202411071815 filed on Sep. 23, 2024, the entirety of which is incorporated herein by reference.

### BACKGROUND

CCR (continuous catalyst regeneration) and fixed-bed catalytic naphtha reforming process unit produce net gas as one of the main products. The net gas contains more than 80 mol % hydrogen with the remainder being C₁-C₇ hydrocarbons. Refineries which do not have hydrocrackers do not require a large amount of hydrogen, so the net gas is typically burned as fuel in fired heaters. Because the hydrogen rich net gas stream contains C₃₊ hydrocarbons, there is a penalty in not recovering the C₃₊ hydrocarbons. The processes also result in higher CO₂ emissions.

One process to recover the hydrocarbons employs a net gas absorber with liquid and vapor chillers and a drier package to chill the net gas and separator liquid to as low as −15° C. to achieve the higher recovery of C₃₊ hydrocarbons from gas phase to liquid phase. This solution has a huge energy penalty due to the power consumption of the refrigeration package.

Another process uses a pressure swing adsorption (PSA) unit and tail gas absorber for higher C₃₊ recovery. However, this solution requires a minimum pressure for the PSA unit of 300 psig and at least 7 barg operating pressure for the tail gas absorber. As a result, the PSA tail gas must be compressed to this pressure which consumes more power. It also involves an expensive customized tail gas compressor design and requires a dedicated plot space. In addition, the PSA unit produces hydrogen product at 99.9 mol %, which is not required if majority of the hydrogen is to be sent to the fuel gas header, instead of sending it to hydrogen chemical consumers like hydrocrackers.

Therefore, there is a need for a lower cost, more effective process for recovering the hydrocarbons from the net gas of CCR and fixed bed catalytic naphtha reforming processes.

### BRIEF DESCRIPTION OF THE DRAWINGS

FIG. 1 illustrates one embodiment of a process for recovering hydrocarbons from the net gas stream of a CCR or fixed bed catalytic naphtha reforming processes.

### DESCRIPTION

The present invention reduces the high energy cost of the refrigeration package by making it much smaller in size and utilizing a membrane separation unit to reject hydrogen preferentially from the net gas and recover the C₃₊ hydrocarbons from a hydrogen depleted retentate stream from the membrane unit using a retentate absorber. The invention eliminates the need for a large tail gas compressor package from the flowscheme, while still achieving the target C₃₊ recovery of 85-90% or more with a smaller refrigeration package in the net gas section and membrane and retentate absorber equipment. It does not require any additional compressors, resulting in a significant reduction in plot space and power consumption.

The invention addresses the carbon emissions issue of many refineries that flare the net gas or use it as fuel gas with only limited net gas being used as chemical consumers. The invention helps to recover more than 85-90% of the C₃₊ hydrocarbons from the hydrogen rich net gas stream without having to use a much larger refrigeration package, or a PSA unit and tail gas compressor. LPG and C₅₊ are high value products, and recovering 90% or more with less equipment, a lower operating cost, and a lower CO₂ footprint is a significant advantage over other processes. Hydrogen from the system will have a purity greater than 96 mol %, which is higher than a simple naphtha reforming recontact section, allowing it to be used as high purity makeup gas. Plants which do not need 99 mol % hydrogen and wants to recover C₃₊ hydrocarbon can adopt this process which has lower capital and operating costs than the refrigeration or PSA and tail gas processes.

The process uses membrane technology and a retentate absorber to recover C₃₊ hydrocarbons efficiently from the CCR and fixed-bed catalytic naphtha reforming process unit net gas stream without the need for any additional compression of the membrane section products.

After net gas compression in the separation zone (typically one or two stages), about 10-15% of the separator liquid is contacted with final stage of the net gas discharge vapor after cooling and chilling to meet the benzene, toluene, and xylenes (BTX) limit of about 200 to 600 volppm to the membrane. The chiller is used only to meet the BTX limit and not for enhanced recovery of C₃₊ hydrocarbons, so the chiller duty is optimized to a smaller size. The target chiller process outlet temperature is 4-15° C. Low temperature heat from the recontact drum liquid and vapor is recovered with the help of vapor and liquid economizers prior to the chiller to reduce the chiller duty.

The net gas section discharge pressure is optimized to supply it to membrane separation zone. The expected net gas battery limit pressure of net gas to the membrane separation zone is 250-300 psig depending on the membrane size, feed to permeate pressure ratio, and the flow to membrane.

The net gas after chilling and chloride treatment, hydrogen is preferentially permeated and separated in the in membrane separation zone. Expected recoveries into the permeate stream is 85-95% for hydrogen, 20% for CH₄, 10% of C₂, 5% for C₃, 5% for C₄ to C₆, 22% for benzene, and 34% for toluene, and 1% for Xylenes.

The retentate (retentate), which is a hydrogen depleted stream and rich in C₁₊ hydrocarbons, is at almost the same pressure as the membrane feed pressure. This stream is recontacted with about 50 to 65% of the separator liquid in a multi-stage absorber column to recover the C₃₊ hydrocarbons effectively from the gas phase to the liquid phase.

The lean vapor from the overhead of absorber column is preferentially chilled in a separate chiller to recover the traces of aromatics which are lost into the vapor phase in the absorber due to reverse mass transfer and their polarity into the gas phase. The chiller is a very small chiller of less than 1 MMBtu/hr duty magnitude for 70,000 BPSD catalyst reforming unit size. It is optional based on the need for recovering the lost aromatics into the vapor phase.

The retentate absorber has sufficient pressure to operate at as high as 250 psig to recover C₃₊ hydrocarbons efficiently from the lean vapor. Since it comes directly from the membrane separation zone without any compression, there is no need of a compressor for this stream.

After the optional chilling, the vapor stream is sent to the fuel gas header without any compression. The vapor stream could be feed into a gas expander to generate power as an optional solution.

The permeate stream from the membrane separation zone is typically available at 100 to 130 psig, and it can be sent to the fuel gas header without any compression.

The permeate stream will typically have a purity of more than 95 mol %, or 96 mol %, or 97 mol %, or 98 mol % hydrogen. Therefore, it can be used for make-up gas to the downstream users, such as naphtha hydrotreaters (NHT), transalkylation units, isomerization units, diesel hydrotreaters, kerosene hydrotreaters, DHT, KHT units. Since the permeate stream is available at a lower pressure of about 100 to 130 psig, an additional stage of compressor would be required in the downstream user's make-up gas compressor machine as an additional cylinder. As it is not a separate compressor, the expected capital cost of this additional cylinder is typically ¼ of a dedicated compressor.

The off gases from the depentanizer or debutanizer columns are recycled to the offgas absorber which is preferably located above the retentate absorber to save plot space. Approximately 25 to 40% of the separator liquid is sent to the absorber and contacted with offgas in a multi-stage absorber column to recover C₃₊ from the offgas before it goes to the fuel gas header. In the overall flow scheme, the net gas compressor discharge pressure is optimized to send to the membrane separation zone. The membrane separates hydrogen from the net gas stream and aids in efficient recovery of C₃₊ hydrocarbons from the hydrogen depleted retentate stream. A smaller chiller in the net gas section is used to meet the BTX limit to membrane separation zone due to the potential concern of BTX degrading the membrane modules. The chiller may share a refrigeration package with an aromatics complex or an existing refrigeration package within the refinery. There is no need for a compressor for any of the product gases leaving the unit with this flow scheme but with only an additional cylinder to the make-up gas service for users require high purity hydrogen as their chemical hydrogen requirement. This results in a significant saving on the equipment cost for the compressor, the operating cost of the compressor, and the plot space for compressor.

This invention is related to UOP CCR and fixed-bed catalytic naphtha reforming process units for both new and revamp units. The above flow scheme shall be implemented for units which require enhanced liquid (C₃₊) hydrocarbon recovery from the reforming net gas when the majority of the hydrogen in the net gas is sent to the fuel gas header, or a process in which 99.9 mol % pure hydrogen is not required and less purity gas is satisfactory, e.g, 95-98 mol % purity gas, in the hydrogen header. A portion of hydrogen gas from the membrane separation zone is utilized in the downstream units as a make-up gas with an additional make-up gas compressor stage, if required.

This process is particularly useful for refineries with a minimal requirement for hydrogen gas for make-up gas and/or that do not have customers for hydrogen and instead want to maximize the liquid recovery of LPG and C₅₊ from the net gas which otherwise will be sent to the fuel gas header.

As mentioned above, the various embodiments contemplated herein relate to apparatuses and methods for the recovery of hydrocarbons products from a reforming effluent. The exemplary embodiments described herein provide a separation zone in fluid communication with a reforming zone to receive a reforming-zone effluent.

As used herein, the term “zone” refers to an area including one or more equipment items and/or one or more sub-zones. Equipment items can include one or more reactors or reactor vessels, scrubbers, strippers, fractionators or distillation columns, absorbers or absorber vessels, adsorber or adsorber vessels, regenerators, heaters, exchangers, coolers/chillers, pipes, pumps, compressors, controllers, membranes, and the like. Additionally, an equipment item can further include one or more zones or sub-zones.

The reforming-zone effluent comprises hydrogen (H₂), C₄⁻ hydrocarbons, and C₅⁺ hydrocarbons including aromatics. As used herein, “Cₓ” means hydrocarbon molecules that have “X” number of carbon atoms, “Cₓ⁺” means hydrocarbon molecules that have “X” and/or more than “X” number of carbon atoms, and “Cₓ⁻” means hydrocarbon molecules that have “X” and/or less than “X” number of carbon atoms.

With these general principles in mind, one or more embodiments of the present invention will be described with the understanding that the following description is not intended to be limiting.

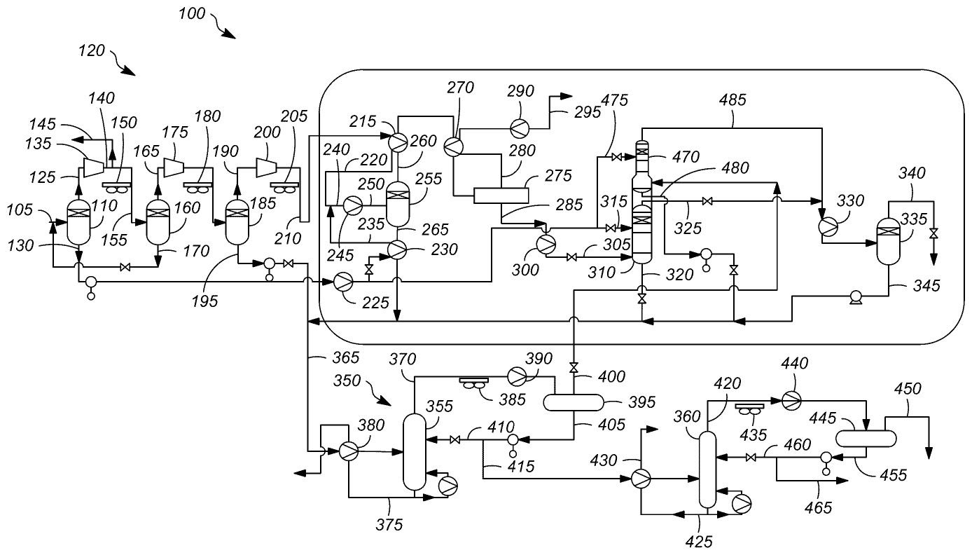

FIG. 1 illustrates one example of a process 100 for recovering hydrogen and hydrocarbons from a process stream.

Although the following uses a reforming effluent stream to as the feed stream 105 to the process for ease of discussion, those of skill in the art understand that the process can be used for any mixed vapor liquid stream comprising hydrocarbon and greater than or equal to 50 mole % hydrogen.

The feed stream 105 from a reforming zone contains H₂, water, H₂S, C₅⁺ hydrocarbons including aromatics, and lighter hydrocarbons such as C₄⁻ hydrocarbons including C₃ and C₄ hydrocarbons. In an exemplary embodiment, the feed stream 105 is a two-phase liquid-gas stream in which H₂ and the lighter hydrocarbons (e.g., C₄⁻ hydrocarbons) are predominantly in the gas phase and the heavier hydrocarbons (e.g., C₅⁺ hydrocarbons including aromatics) are predominantly in the liquid phase. In one embodiment, the feed stream 105 has a temperature of from about 35 to about 60° C. and, independently, a pressure of from about 69 to about 830 kPa gauge (10 and 120 psig).

The feed stream 105 is introduced to a separation vessel 110 in the first separation zone 120. In the separation vessel 110, the feed stream 105 is separated into a gas phase stream 125 and a liquid hydrocarbon stream 130. In an exemplary embodiment, the gas phase stream 125 comprises H₂, C₆⁻ hydrocarbons, and impurities such as carbon monoxide and/or nitrogen, and the liquid hydrocarbon stream 130 comprises C₅⁺ hydrocarbons including aromatics. In one example, the gas phase stream 125 comprises H₂ present in an amount of from about 80 to about 90 mole %, C₁ hydrocarbons present in an amount of about 2 to about 5 mole %, C₂ hydrocarbons present in an amount of from about 2 to about 5 mole %, C₃ hydrocarbons present in an amount of from about 2 to about 4 mole %, C₄ hydrocarbons present in an amount of from about 1.5 to about 2.5 mole %, and possibly some C₅⁺ hydrocarbons. In another example, the liquid hydrocarbon stream 130 comprises C₅⁺ hydrocarbons present in an amount of from about 90 to about 99.9 mole % and possibly some C₄⁻ hydrocarbons and H₂.

In an exemplary embodiment, the separation vessel 110 is operated at a temperature of from about 35 to about 60° C. (95 to 122° F.). and, independently, a pressure of from about 70 to about 830 kPa gauge (10 to 120 psig).

The gas phase stream 125 is passed to a compressor 135 to form a compressed gas phase stream 140. A portion 145 of the compressed gas phase stream 140 may be used as a recycle gas phase stream 145 to a reforming reaction zone (not shown), for example. The rest of the compressed gas phase stream 140 is passed to a cooler 150. In the cooler 150, the compressed gas phase stream 140 is partially cooled to form a partially cooled, compressed gas phase stream 155. In an exemplary embodiment, the partially cooled, compressed gas phase stream 155 has a temperature of from about 30 to about 65° C. (86 to 149° F.) and, independently, a pressure of from about 410 to about 1,724 kPa gauge (60 to 250 psig).

The partially cooled, compressed gas phase stream 155 may be passed to a vessel 160, which may be a suction drum. In the vessel 160, the partially cooled, compressed gas phase stream 155 is separated into a first stage vapor stream 165 and a first stage liquid stream 170. The first stage liquid stream 170 may be used as a recycle stream to the separation vessel 110. The first stage vapor stream 165 is compressed in a compressor 175, cooled in a cooler 180, and passed to a second vessel 185, which may be a discharge drum.

In the second vessel 185, the first stage vapor stream 165 is separated into a second stage vapor stream 190 and a second stage liquid stream 195. The second stage vapor stream 190 is compressed in another compressor 200 and cooled in another cooler 205. The cooled second stage vapor stream 210 is further cooled in a vapor economizer 215 forming a second cooled second stage vapor stream 220. Any number of separation vessels and stages of compression may be used, and the depicted arrangement is merely exemplary.

The liquid hydrocarbon stream 130 from the separation vessel 110 is cooled in liquid cooler 225 and further cooled in liquid economizer 230. The cooled liquid hydrocarbon stream 235 is combined with the second cooled second stage vapor stream 220 forming combined stream 240. The combined stream 240 is chilled in chiller 245, and the chilled combined stream 250 is passed to a third vessel 255, which can be a recontact drum.

In the third vessel 255, the chilled combined stream 250 is separated into a membrane vapor feed stream 260, or a net gas phase stream comprising C₆⁻ hydrocarbons and H₂, and a hydrocarbon enriched liquid stream 265. The membrane vapor feed stream 260 is heated in the vapor economizer 215, further heated in the membrane feed-permeate exchanger 270, and sent to the membrane separation zone 275.

The membrane vapor feed stream 260 is separated in the membrane separation zone 275 into hydrogen rich permeate stream 280 and retentate stream 285.

The temperature of the membrane vapor feed stream 260 is in the range of 15.6 and 71.1° C. (60 and 160° F.) and a pressure in the range of approximately 1,379 and 2,413 kPa gauge (200 to 350 psig).

The hydrogen rich permeate stream 280, which comprises greater than 95 mole % hydrogen, is cooled in the membrane feed-permeate exchanger 270, and further cooled in exchanger 290. The cooled hydrogen rich permeate stream 295 is recovered for use in processes not requiring high purity hydrogen.

The retentate stream 285 comprising hydrocarbons is cooled in retentate cooler 300, and the cooled retentate stream 305 is sent to the retentate absorber vessel 310. The cooled retentate stream 305 enters the lower part of the retentate absorber vessel 310, which may have 20 to thirty trays, for example. A second portion 315 of the liquid hydrocarbon stream 130 enters the upper portion of the retentate absorber vessel 310 and falls downwardly for countercurrent contact with the rising cooled retentate stream 305. During contact in the retentate absorber vessel 310, C₃/C₄ hydrocarbons from the cooled retentate stream 305 are extracted and/or absorbed to the second portion 315 of the liquid hydrocarbon stream 130 to form a hydrocarbon enriched liquid stream 320. In addition to C₃/C₄ hydrocarbons, the hydrocarbon enriched liquid stream 320 may include C₅⁺ hydrocarbons.

A hydrocarbon lean vapor stream 325, which comprises H₂, as well as C₂⁻ hydrocarbons, is also recovered from the retentate absorber vessel 310. The hydrocarbon lean vapor stream 325 is cooled in an offgas chiller 330 and sent to offgas knockout drum 335 where it is separated into an offgas hydrocarbon lean vapor stream 340 and an offgas hydrocarbon enriched liquid stream 345.

One or more of the hydrocarbon enriched liquid stream 320 from the retentate absorber vessel 310, the hydrocarbon enriched liquid stream 265 from the third vessel 255, the offgas hydrocarbon enriched liquid stream 345 from the offgas knockout drum 335, and/or the second stage liquid stream 195 from the second vessel 185 are combined and sent to the fractionation zone 350 for recovery of the hydrocarbons. The fractionation zone 350 may comprise one or more fractionation columns as is known in the art.

For example, the fractionation zone 350 may comprise a depentanizer column 355 and a debutanizer column 360. The combined hydrocarbon enriched stream 365 is separated in the depentanizer column 355 into a depentanizer overhead stream 370 comprising C₅₋ hydrocarbons and a depentanizer bottom stream 375 comprising C₆₊ hydrocarbons. The depentanizer bottom stream 375 can be heat exchanged with the combined hydrocarbon enriched stream 365 in heat exchanger 380 and recovered.

The depentanizer overhead stream 370 is cooled in cooler 385 and heat exchanger 390 and sent to depentanizer receiver 395 where it is separated into a depentanizer overhead vapor stream 400 and depentanizer overhead liquid stream 405. The depentanizer overhead liquid stream 405 can be divided into a first portion 410 which is refluxed to the depentanizer column 355 and a second portion 415 which sent to the debutanizer column 360.

The second portion 415 of the depentanizer overhead liquid stream 405 is separated in the debutanizer column 360 into a debutanizer overhead stream 420 comprising C₄₋ hydrocarbons and a debutanizer bottom stream 425 comprising C₅ hydrocarbons. The debutanizer bottom stream 425 can be heat exchanged with the second portion 415 of the depentanizer overhead liquid stream 405 in heat exchanger 430 and recovered.

The debutanizer overhead stream 420 is cooled in cooler 435 and heat exchanger 440 and sent to debutanizer receiver 445 where it is separated into a debutanizer overhead vapor stream 450 and debutanizer overhead liquid stream 455. The debutanizer overhead liquid stream 455 can be divided into a first portion 460 which is refluxed to the debutanizer column 360 and a second portion 465 which is recovered as LPG product.

The depentanizer overhead vapor stream 400 is sent to the lower portion of an offgas absorber vessel 470, which may have 10 to 20 trays, for example. A third portion 475 of the liquid hydrocarbon stream 130 enters the upper portion of the offgas absorber vessel 470 and falls downwardly for countercurrent contact with the rising depentanizer overhead vapor stream 400. During the contact, C₃/C₄ hydrocarbons from the depentanizer overhead vapor stream 400 are extracted and/or absorbed to the third portion 475 of the liquid hydrocarbon stream 130 to form a third hydrocarbon enriched liquid stream 480. In addition to C₃/C₄ hydrocarbons, the third hydrocarbon enriched liquid stream 480 may include C₅₊ hydrocarbons. The third hydrocarbon enriched liquid stream 480 can be combined with one or more of the hydrocarbon enriched liquid stream 320 from the retentate absorber vessel 310, the hydrocarbon enriched liquid stream 265 from the third vessel 255, the offgas hydrocarbon enriched liquid stream 345 from the offgas knockout drum 335, and/or the second stage liquid stream 195 from the second vessel 185 and sent to the fractionation zone 350.

The offgas stream 485, which comprises C₂₋ and left out C₃/C₄ and traces of C₅₊ hydrocarbons, from the offgas absorber vessel 470 is chilled in offgas chiller 330 and separated in the offgas knockout drum 335. It can be combined with the hydrocarbon lean vapor stream 325 from the retentate absorber vessel 310 for chilling and separation.

### Specific Embodiments

While the following is described in conjunction with specific embodiments, it will be understood that this description is intended to illustrate and not limit the scope of the preceding description and the appended claims.

A first embodiment of the invention is a process for recovering hydrogen and hydrocarbons from a process stream, the process comprising separating the process stream comprising greater than or equal to 30 mole % hydrogen and C₁ to C₆₊ hydrocarbons in a first separation zone to form a net vapor stream comprising C₆₋ hydrocarbons and H₂, and a liquid hydrocarbon stream comprising C₅₊ hydrocarbons including aromatics, and optionally a second liquid hydrocarbon stream comprising C₅₊ hydrocarbons including aromatics; separating the net vapor stream in a membrane separation zone to form a hydrogen rich permeate stream comprising greater than 95 mole % hydrogen and a retentate stream comprising the hydrocarbons; absorbing hydrocarbons from the retentate stream using a portion of the liquid hydrocarbon stream from the first separation zone in a retentate absorption zone to form a hydrocarbon lean vapor stream comprising hydrogen and a hydrocarbon enriched liquid stream; separating the hydrocarbon enriched liquid stream in a hydrocarbon separation zone into at least one hydrocarbon product stream; and optionally recovering the hydrogen rich permeate stream. An embodiment of the invention is one, any or all of prior embodiments in this paragraph up through the first embodiment in this paragraph further comprising chilling the net vapor stream and a second portion of the liquid hydrocarbon stream from the first separation zone; separating the chilled stream in a recontact drum into a membrane vapor feed stream and a second hydrocarbon enriched liquid stream; and passing the membrane vapor feed stream to the membrane separation zone. An embodiment of the invention is one, any or all of prior embodiments in this paragraph up through the first embodiment in this paragraph further comprising combining the net vapor stream and the first portion of the liquid hydrocarbon stream before chilling the net vapor stream and the first portion of the liquid hydrocarbon stream. An embodiment of the invention is one, any or all of prior embodiments in this paragraph up through the first embodiment in this paragraph further comprising cooling the net vapor stream or the portion of the liquid hydrocarbon stream or both before chilling the net vapor stream or the portion of the liquid hydrocarbon stream. An embodiment of the invention is one, any or all of prior embodiments in this paragraph up through the first embodiment in this paragraph wherein cooling the net vapor stream or the portion of the liquid hydrocarbon stream or both comprises cooling the net vapor stream in a vapor economizer or the portion of the liquid hydrocarbon stream in a liquid economizer or both. An embodiment of the invention is one, any or all of prior embodiments in this paragraph up through the first embodiment in this paragraph wherein the hydrocarbon separation zone comprises a depentanizer column, and wherein separating the hydrocarbon enriched liquid stream into at least one hydrocarbon product stream comprises separating the hydrocarbon enriched liquid stream from the retentate absorption zone in the depentanizer column into a depentanizer overhead stream comprising C₅₋ hydrocarbons and a depentanizer bottom stream comprising C₆₊ hydrocarbons; recovering the depentanizer bottom stream. An embodiment of the invention is one, any or all of prior embodiments in this paragraph up through the first embodiment in this paragraph further comprising condensing the depentanizer overhead stream; separating the condensed depentanizer overhead stream into a depentanizer overhead vapor stream and a depentanizer overhead liquid stream. An embodiment of the invention is one, any or all of prior embodiments in this paragraph up through the first embodiment in this paragraph further comprising absorbing hydrocarbons from the depentanizer overhead vapor stream in an offgas absorption zone using a third portion of the liquid hydrocarbon stream from the first separation zone forming a hydrocarbon lean offgas vapor stream and a third hydrocarbon enriched liquid stream; combining the third hydrocarbon enriched liquid stream from the offgas absorption zone and the second hydrocarbon enriched liquid stream from the recontact drum with the hydrocarbon enriched liquid stream from the retentate absorption zone into a combined hydrocarbon enriched liquid stream; wherein separating the hydrocarbon enriched liquid stream comprises separating the combined hydrocarbon enriched liquid stream. An embodiment of the invention is one, any or all of prior embodiments in this paragraph up through the first embodiment in this paragraph further comprising chilling the hydrocarbon lean offgas vapor stream; separating the hydrocarbon lean offgas vapor stream in an offgas knockout drum into a fourth hydrocarbon enriched liquid stream and a second hydrocarbon lean offgas vapor stream; and combining the fourth hydrocarbon enriched liquid stream with the combined hydrocarbon enriched liquid stream. An embodiment of the invention is one, any or all of prior embodiments in this paragraph up through the first embodiment in this paragraph wherein the hydrocarbon separation zone further comprises a debutanizer column, and further comprising separating the depentanizer overhead liquid stream in the debutanizer column into a debutanizer overhead liquid stream comprising C₄₋ hydrocarbons and a debutanizer bottom stream comprising C₅ hydrocarbons; recovering the debutanizer overhead stream; and recovering the debutanizer bottom stream. An embodiment of the invention is one, any or all of prior embodiments in this paragraph up through the first embodiment in this paragraph further comprising; passing the second liquid hydrocarbon stream from the first separation zone to the hydrocarbon separation zone. An embodiment of the invention is one, any or all of prior embodiments in this paragraph up through the first embodiment in this paragraph further comprising chilling the hydrocarbon lean vapor stream from the retentate absorption zone; separating the hydrocarbon lean vapor stream in an offgas knockout drum into a fifth hydrocarbon enriched liquid stream and a second hydrocarbon lean vapor stream.

A second embodiment of the invention is a process for recovering hydrogen and hydrocarbons from a process stream, the process comprising separating the process stream comprising greater than or equal to 30 mole % hydrogen and C₁ to C₆₊ hydrocarbons in a first separation zone to form a net vapor stream comprising C₆₋ hydrocarbons and H₂, and a liquid hydrocarbon stream comprising C₅₊ hydrocarbons including aromatics, and optionally a second liquid hydrocarbon stream comprising C₅₊ hydrocarbons including aromatics; separating the net vapor stream in a membrane separation zone to form a hydrogen rich permeate stream comprising greater than 95 mole % hydrogen and a retentate stream comprising the hydrocarbons; absorbing hydrocarbons from the retentate stream using a portion of the liquid hydrocarbon stream from the first separation zone in a retentate absorption zone to form a hydrocarbon lean vapor stream comprising hydrogen and a hydrocarbon enriched liquid stream; separating the hydrocarbon enriched liquid stream in a hydrocarbon separation zone comprising a depentanizer column into at least a depentanizer overhead stream comprising C₅₋ hydrocarbons and a depentanizer bottom stream comprising C₆₊ hydrocarbons; recovering the depentanizer bottom stream; condensing the depentanizer overhead stream; separating the condensed depentanizer overhead stream into a depentanizer overhead vapor stream and a depentanizer overhead liquid stream; and optionally recovering the hydrogen rich permeate stream. An embodiment of the invention is one, any or all of prior embodiments in this paragraph up through the second embodiment in this paragraph further comprising absorbing hydrocarbons from the depentanizer overhead vapor stream in an offgas absorption zone using a third portion of the liquid hydrocarbon stream from the first separation zone forming a hydrocarbon lean offgas vapor stream and a third hydrocarbon enriched liquid stream; combining the third hydrocarbon enriched liquid stream from the offgas absorption zone and the second hydrocarbon enriched liquid stream from the recontact drum with the hydrocarbon enriched liquid stream from the retentate absorption zone into a combined hydrocarbon enriched liquid stream; wherein separating the hydrocarbon enriched liquid stream comprises separating the combined hydrocarbon enriched liquid stream. An embodiment of the invention is one, any or all of prior embodiments in this paragraph up through the second embodiment in this paragraph further comprising chilling the hydrocarbon lean offgas vapor stream; separating the hydrocarbon lean offgas vapor stream in an offgas knockout drum into a fourth hydrocarbon enriched liquid stream and a second hydrocarbon lean offgas vapor stream; and combining the fourth hydrocarbon enriched liquid stream with the combined hydrocarbon enriched liquid stream. An embodiment of the invention is one, any or all of prior embodiments in this paragraph up through the second embodiment in this paragraph further comprising combining the net vapor stream and a second portion of the liquid hydrocarbon stream forming a combined stream; chilling the combined stream forming a chilled stream; separating the chilled stream in a recontact drum into a membrane vapor feed stream and a second hydrocarbon enriched liquid stream; and passing the membrane vapor feed stream to the membrane separation zone. An embodiment of the invention is one, any or all of prior embodiments in this paragraph up through the second embodiment in this paragraph further comprising cooling the net vapor stream in a vapor economizer or cooling the portion of the liquid hydrocarbon stream in a liquid economizer or both before chilling the net vapor stream or the portion of the liquid hydrocarbon stream. An embodiment of the invention is one, any or all of prior embodiments in this paragraph up through the second embodiment in this paragraph wherein the hydrocarbon separation zone further comprises a debutanizer column, and further comprising separating the depentanizer overhead liquid stream in the debutanizer column into a debutanizer overhead liquid stream comprising C₄₋ hydrocarbons and a debutanizer bottom stream comprising C₅ hydrocarbons; recovering the debutanizer overhead stream; and recovering the debutanizer bottom stream. An embodiment of the invention is one, any or all of prior embodiments in this paragraph up through the second embodiment in this paragraph further comprising; passing the second liquid hydrocarbon stream from the first separation zone to the hydrocarbon separation zone. An embodiment of the invention is one, any or all of prior embodiments in this paragraph up through the second embodiment in this paragraph further comprising chilling the hydrocarbon lean vapor stream from the retentate absorption zone; separating the hydrocarbon lean vapor stream in an offgas knockout drum into a fifth hydrocarbon enriched liquid stream and a second hydrocarbon lean vapor stream.

Without further elaboration, it is believed that using the preceding description that one skilled in the art can utilize the present invention to its fullest extent and easily ascertain the essential characteristics of this invention, without departing from the spirit and scope thereof, to make various changes and modifications of the invention and to adapt it to various usages and conditions. The preceding preferred specific embodiments are, therefore, to be construed as merely illustrative, and not limiting the remainder of the disclosure in any way whatsoever, and that it is intended to cover various modifications and equivalent arrangements included within the scope of the appended claims.

In the foregoing, all temperatures are set forth in degrees Celsius and, all parts and percentages are by weight, unless otherwise indicated.

What is claimed is:

1. A process for recovering hydrogen and hydrocarbons from a process stream, the process comprising:
separating the process stream comprising greater than or equal to 30 mole % hydrogen and C₁ to C₆₊ hydrocarbons in a first separation zone to form a net vapor stream comprising C₆₋ hydrocarbons and H₂, and a liquid hydrocarbon stream comprising C₅₊ hydrocarbons including aromatics, and optionally a second liquid hydrocarbon stream comprising C₅₊ hydrocarbons including aromatics;
separating the net vapor stream in a membrane separation zone to form a hydrogen rich permeate stream comprising greater than 95 mole % hydrogen and a retentate stream comprising the hydrocarbons;
absorbing hydrocarbons from the retentate stream using a portion of the liquid hydrocarbon stream from the first separation zone in a retentate absorption zone to form a hydrocarbon lean vapor stream comprising hydrogen and a hydrocarbon enriched liquid stream;
separating the hydrocarbon enriched liquid stream in a hydrocarbon separation zone into at least one hydrocarbon product stream; and
optionally recovering the hydrogen rich permeate stream.

2. The process of claim 1 further comprising:
chilling the net vapor stream and a second portion of the liquid hydrocarbon stream from the first separation zone;
separating the chilled stream in a recontact drum into a membrane vapor feed stream and a second hydrocarbon enriched liquid stream; and
passing the membrane vapor feed stream to the membrane separation zone.

3. The process of claim 2 further comprising:
combining the net vapor stream and the first portion of the liquid hydrocarbon stream before chilling the net vapor stream and the first portion of the liquid hydrocarbon stream.

4. The process of claim 3 further comprising:
cooling the net vapor stream or the portion of the liquid hydrocarbon stream or both before chilling the net vapor stream or the portion of the liquid hydrocarbon stream.

5. The process of claim 4 wherein cooling the net vapor stream or the portion of the liquid hydrocarbon stream or both comprises cooling the net vapor stream in a vapor economizer or the portion of the liquid hydrocarbon stream in a liquid economizer or both.

6. The process of claim 1 wherein the hydrocarbon separation zone comprises a depentanizer column, and wherein separating the hydrocarbon enriched liquid stream into at least one hydrocarbon product stream comprises:
separating the hydrocarbon enriched liquid stream from the retentate absorption zone in the depentanizer column into a depentanizer overhead stream comprising C₅₋ hydrocarbons and a depentanizer bottom stream comprising C₆₊ hydrocarbons; and
recovering the depentanizer bottom stream.

7. The process of claim 6 further comprising:
condensing the depentanizer overhead stream; and
separating the condensed depentanizer overhead stream into a depentanizer overhead vapor stream and a depentanizer overhead liquid stream.

8. The process of claim 7 further comprising:
absorbing hydrocarbons from the depentanizer overhead vapor stream in an offgas absorption zone using a third portion of the liquid hydrocarbon stream from the first separation zone forming a hydrocarbon lean offgas vapor stream and a third hydrocarbon enriched liquid stream;
combining the third hydrocarbon enriched liquid stream from the offgas absorption zone and the second hydrocarbon enriched liquid stream from the recontact drum with the hydrocarbon enriched liquid stream from the retentate absorption zone into a combined hydrocarbon enriched liquid stream; and
wherein separating the hydrocarbon enriched liquid stream comprises separating the combined hydrocarbon enriched liquid stream.

9. The process of claim 8 further comprising:
chilling the hydrocarbon lean offgas vapor stream;
separating the hydrocarbon lean offgas vapor stream in an offgas knockout drum into a fourth hydrocarbon enriched liquid stream and a second hydrocarbon lean offgas vapor stream; and
combining the fourth hydrocarbon enriched liquid stream with the combined hydrocarbon enriched liquid stream.

10. The process of claim 7 wherein the hydrocarbon separation zone further comprises a debutanizer column, and further comprising:
separating the depentanizer overhead liquid stream in the debutanizer column into a debutanizer overhead liquid stream comprising C₄₋ hydrocarbons and a debutanizer bottom stream comprising C₅ hydrocarbons;
recovering the debutanizer overhead stream; and
recovering the debutanizer bottom stream.

11. The process of claim 1 further comprising;
passing the second liquid hydrocarbon stream from the first separation zone to the hydrocarbon separation zone.

12. The process of claim 1 further comprising:
chilling the hydrocarbon lean vapor stream from the retentate absorption zone; and
separating the hydrocarbon lean vapor stream in an offgas knockout drum into a fifth hydrocarbon enriched liquid stream and a second hydrocarbon lean vapor stream.

13. A process for recovering hydrogen and hydrocarbons from a process stream, the process comprising:
separating the process stream comprising greater than or equal to 30 mole % hydrogen and C₁ to C₆₊ hydrocarbons in a first separation zone to form a net vapor stream comprising C₆₋ hydrocarbons and H₂, and a liquid hydrocarbon stream comprising C₅₊ hydrocarbons including aromatics, and optionally a second liquid hydrocarbon stream comprising C₅₊ hydrocarbons including aromatics;
separating the net vapor stream in a membrane separation zone to form a hydrogen rich permeate stream comprising greater than 95 mole % hydrogen and a retentate stream comprising the hydrocarbons;
absorbing hydrocarbons from the retentate stream using a portion of the liquid hydrocarbon stream from the first separation zone in a retentate absorption zone to form a hydrocarbon lean vapor stream comprising hydrogen and a hydrocarbon enriched liquid stream;
separating the hydrocarbon enriched liquid stream in a hydrocarbon separation zone comprising a depentanizer column into at least a depentanizer overhead stream comprising C₅₋ hydrocarbons and a depentanizer bottom stream comprising C₆₊ hydrocarbons;
recovering the depentanizer bottom stream;
condensing the depentanizer overhead stream;
separating the condensed depentanizer overhead stream into a depentanizer overhead vapor stream and a depentanizer overhead liquid stream; and
optionally recovering the hydrogen rich permeate stream.

14. The process of claim 13 further comprising:
absorbing hydrocarbons from the depentanizer overhead vapor stream in an offgas absorption zone using a third portion of the liquid hydrocarbon stream from the first separation zone forming a hydrocarbon lean offgas vapor stream and a third hydrocarbon enriched liquid stream;
combining the third hydrocarbon enriched liquid stream from the offgas absorption zone and the second hydrocarbon enriched liquid stream from the recontact drum with the hydrocarbon enriched liquid stream from the retentate absorption zone into a combined hydrocarbon enriched liquid stream; and
wherein separating the hydrocarbon enriched liquid stream comprises separating the combined hydrocarbon enriched liquid stream.

15. The process of claim 14 further comprising:
chilling the hydrocarbon lean offgas vapor stream;
separating the hydrocarbon lean offgas vapor stream in an offgas knockout drum into a fourth hydrocarbon enriched liquid stream and a second hydrocarbon lean offgas vapor stream; and
combining the fourth hydrocarbon enriched liquid stream with the combined hydrocarbon enriched liquid stream.

16. The process of claim 13 further comprising:
combining the net vapor stream and a second portion of the liquid hydrocarbon stream forming a combined stream;
chilling the combined stream forming a chilled stream;
separating the chilled stream in a recontact drum into a membrane vapor feed stream and a second hydrocarbon enriched liquid stream; and
passing the membrane vapor feed stream to the membrane separation zone.

17. The process of claim 16 further comprising:
cooling the net vapor stream in a vapor economizer or cooling the portion of the liquid hydrocarbon stream in a liquid economizer or both before chilling the net vapor stream or the portion of the liquid hydrocarbon stream.

18. The process of claim 13 wherein the hydrocarbon separation zone further comprises a debutanizer column, and further comprising:
separating the depentanizer overhead liquid stream in the debutanizer column into a debutanizer overhead liquid stream comprising C₄₋ hydrocarbons and a debutanizer bottom stream comprising C₅ hydrocarbons;
recovering the debutanizer overhead stream; and
recovering the debutanizer bottom stream.

19. The process of claim 1 further comprising;
passing the second liquid hydrocarbon stream from the first separation zone to the hydrocarbon separation zone.

20. The process of claim 1 further comprising:
chilling the hydrocarbon lean vapor stream from the retentate absorption zone; and
separating the hydrocarbon lean vapor stream in an offgas knockout drum into a fifth hydrocarbon enriched liquid stream and a second hydrocarbon lean vapor stream.
